(19) United States
(12) Patent Application Publication
(10) Pub. No.: US 2011/0206790 A1
(43) Pub. Date: Aug. 25, 2011

(54) Antimicrobial Foamable Soaps

(75) Inventor: Larry Weiss, San Francisco, CA (US)

(73) Assignee: OHSO CLEAN, INC

(21) Appl. No.: 13/126,628

(22) PCT Filed: Oct. 29, 2009
(86) PCT No.: PCT/US09/62527

§ 371 Date: Apr. 28, 2011

Related U.S. Application Data
(60) Provisional application No. 61/109,721, filed on Oct. 30, 2008.

(51) Int. Cl. A61K 36/53; A01N 65/22; A01P 15/00; A61P 17/02

(52) U.S. Cl. 424/745

(57) ABSTRACT

Environmentally-friendly, natural essential oil based foamable compositions can be used as antimicrobial substances. The foamable compositions contain thyme oil as the sole or principal antimicrobial agent. The foamable compositions are topically applied and may be used as a preventative and/or a therapeutic. The antimicrobial foamable compositions are highly efficacious against methicillin-resistant Staphylococcus aureus (MRSA).

### CROSS-REFERENCE TO RELATED APPLICATIONS

This application claims priority to and benefit under 35 U.S.C. §119(e) to U.S. Provisional Application No. 61/109,721, filed Oct. 30, 2008, the disclosure of which is herein expressly incorporated by reference in its entirety.

### FIELD OF INVENTION

The invention generally relates to antimicrobial foamable compositions for the treatment and prevention of skin infections. More particularly, the invention relates to topical antimicrobial foamable compositions containing thyme oil for the treatment and prevention of bacterial infections and drug-resistant bacterial infections, such as methicillin-resistant Staphylococcus aureus (MRSA).

### BACKGROUND OF THE INVENTION

Staphylococcus aureus, often referred to simply as “Staph,” is a bacterial strain that is found normally on the skin or in the nose of roughly one third of the population. Staph infections are one of the most common causes of skin infections in the United States. Most Staph infections are minor and can be treated with antibiotics. However, Staph can also cause serious infections of the skin and soft tissue, bloodstream infections, and pneumonia.

Over time, bacteria may become resistant to certain types of antibiotics. Methicillin-resistant Staphylococcus aureus (MRSA) is a strain of Staph resistant to the beta-lactam antibiotics (e.g., penicillins and cephalosporins), the most widely used class of antibiotics to treat bacterial infections. While other synthetic and semi-synthetic antibiotics (e.g., vancomycin and oxazolidines) are currently useful for treating MRSA, there have been several reports of emerging resistance to these agents as well. MRSA infections may occur among individuals in hospitals and healthcare facilities (healthcareassociated MRSA (HA-MRSA)), or in individuals in the greater community (community-associated MRSA (CA-MRSA)). The majority of drug-resistant Staph infections are healthcare-associated. The occurrence of MRSA infections, in particular, in hospitalized patients has steadily increased from 2% in 1974 to 22% in 1995 and up to 64% as of 2004. See, Klevens et al., Changes in the Epidemiology of Methicillin-Resistant Staphylococcus aureus in Intensive Care Units in US Hospitals, 1992-2003. Clinical Infectious Diseases, 42:389-91, 2006. There are an estimated 292,000 hospitalizations attributed to Staph infections annually. Of these, approximately 126,000 hospitalizations are specifically associated with MRSA. Approximately 19,000 deaths each year are attributed to MRSA infection.

Alternative agents to prevent or treat microbial infections, particularly drug-resistant bacterial infections, are in high demand. Products that are both natural, such as essential plant oils, and effective are especially desirable candidates.

Accordingly, provided herein is a green and environmentally-friendly antimicrobial foamable composition. In particular, the principal or sole antimicrobial agent in the foamable composition is thyme oil. The antimicrobial foamable compositions described herein provide many advantages over conventionally available antimicrobial foams employing synthetic and semi-synthetic antimicrobial compounds, including non-toxicity to individuals, fewer adverse side effects, increased efficacy, particularly against MRSA, and a lower incidence of failure thereby causing fewer resistant infections as a result of treatment.

### SUMMARY OF THE INVENTION

The invention provides a green and environmentally antimicrobial foamable composition for killing MRSA on a surface contaminated therewith, and methods for killing MRSA on a surface contaminated therewith. The invention may be implemented in a number of ways.

According to one aspect of the invention, the foamable composition comprises an antimicrobial amount of thyme oil of about 0.01% w/v to about 0.5% w/v, a surfactant and a source of divalent copper ions. The composition kills greater than 99.9% or greater than 99.99% of MRSA in 15 seconds when applied to the contaminated surface. In one embodiment, the composition contains about 0.01% w/v to about 0.2% w/v of thyme oil. A copper peptide complex, such as copper PCA, may be the source of the divalent copper ion. The surfactant may include anionic or nonionic surfactants, which may be selected from the non-limiting group of alkyl polyglucoside surfactants, sodium lauryl sulfate, and sodium laureth sulfate. Particular efficacy is associated with the use of an alkyl polyglucoside surfactant, which may be either an anionic or nonionic surfactant. Preferably, the surfactant is a biobased surfactant, which is a surfactant that is not principally derived from a petrochemical feedstock.

The foamable composition may also contain, in addition to thyme oil, origanum oil, which may be present in an amount of about 0.01% w/v to about 0.2% w/v. In addition, the composition may further contain thymol crystals, which may be present in an amount of about 0.01% w/v to about 0.05% w/v.

The foamable composition may further contain at least one fragrance, which may be selected from the non-limiting group of blood orange, vanilla, lavender, ginger, bergamot, spearmint, and lime. The composition may also contain an antioxidant, such as white tea extract.

In a preferred embodiment, the topical foamable composition for killing MRSA on a surface contaminated therewith comprises 0.016% w/v thyme oil, 0.016% w/v origanum oil, 0.032% w/v botanical thymol crystals, 3.0% v/v alkyl polyglucoside surfactant, 0.008% w/v copper PCA, and water to 100%. The composition kills greater than 99.9% or greater than 99.99% of MRSA in 15 seconds when applied to the contaminated surface. The composition may further comprise one or more components selected from white tea, citric acid, sodium citrate, co-surfactant, natural moisturizer, and aloe vera.

According to another aspect of the invention, a method for killing MRSA on a surface contaminated therewith is provided. The method includes applying to the surface a foamable composition comprising an antimicrobial amount of thyme oil of about 0.01% w/v to about 0.5% w/v, a surfactant and a source of divalent copper ions. Further, the method includes exposing the surface to the composition for an exposure time sufficient to kill MRSA. The surface may be mammalian skin, in particular human skin. The composition may be applied to an open wound. The composition may be applied for an exposure time of, for example, at least 15 seconds, at least 5 minutes, or at least an hour. The composition may be applied at least once a day. The composition kills greater than 99.9% or greater than 99.99% of MRSA in 15 seconds.

According to an aspect of the invention, a kit is provided. The kit comprises an antimicrobial foamable composition for killing MRSA on a surface contaminated therewith, the composition comprising an antimicrobial amount of thyme oil of about 0.01% w/v to about 0.5% w/v, a surfactant and a source of divalent copper ions. Further, the kit comprises a dispenser.

Additional features, advantages, and embodiments of the invention may be set forth or apparent from the following detailed description, drawings, and claims. Moreover, both the foregoing summary of the invention and the following detailed description are exemplary and intended to provide further explanation without limiting the scope of the invention as claimed.

### DETAILED DESCRIPTION OF THE INVENTION

The foamable compositions described herein are environmentally friendly and green compositions for killing MRSA on a contaminated surface. The foamable compositions employ a low concentration of thyme oil as the principal or sole antimicrobial agent, a surfactant and a source of divalent copper ions. The foams may be used to prevent or treat skin infections, in particular MRSA infections. Symptoms of a skin infection can include lesions, sores, rashes, blisters, abscesses, or cracking of the skin, all of which can by accompanied by discoloration, inflammation, swelling, or pain. The foams may treat infections by reducing the duration of the symptoms, reducing the severity of the symptoms, or both. Additionally, the foams may cure the infection and/or eliminate the symptoms of the infection.

As used herein, the terms “foamable” and “foam” refer to a substance that is made by forming and trapping gas bubbles in a liquid. A foam may be formed by injecting air into a liquid foamable composition and trapping the air. In particular, a foam can be formed by dispensing the antimicrobial compositions described herein from a container (e.g., bottle or pump) such that the composition is mixed with gas bubbles, and the bubbles are trapped in the composition. Conventional devices for generating a foam from a liquid can be employed with the compositions and methods of the present invention.

As used herein, the terms “natural” and “botanical” refer to substances that are derived, either whole or in part, from natural (e.g., plant) sources. These substances have minimal environmental impact and require minimal non-renewable inputs when the entire life cycle of the substance is considered. As used herein, an “all-natural” substance is obtained from a botanical source or with the aid of biological processes. For example, “natural thymol” refers to thymol obtained from a plant whereas “synthetic thymol” refers to thymol that is chemically synthesized from petroleum. The foamable compositions described herein can contain “all-natural”, “botanical” products. “All-natural” does not exclude various separation processes such as extraction, distilling, etc. or minimal synthetic (i.e., semi-synthetic) transformations of natural products. It is intended to exclude products that have been solely chemically created by man. The products within the scope of the invention are preferably natural based products.

As used herein, the term “environmentally friendly” is used to refer to compounds and products designed to inflict minimal harm on the environment. “Environmentally-friendly” ingredients and chemicals may be used in place of standard products containing chemically-reactive and toxic compounds to avoid or minimize the adverse effects on the environment. As used herein, the term “green” is used to refer to compounds and products that minimize human illnesses as well as minimize damage to the environment as a result of exposure to the compound or product. The EPA has provided principles directed to green chemistry, which include (i) the design of safer chemicals and products that are fully effective, yet have little or no toxicity, and (ii) the design of less hazardous chemical syntheses. Both of these principles apply to the current invention. The term “green” also includes naturally-derived products, such as the thyme oil of the present invention, that have undergone little chemical modification or no chemical modification. Thus, the foamable compositions described herein are “environmentally friendly,” “green” products.

As used herein, the term “antimicrobial” refers to a substance that kills or inhibits the growth of microbes such as bacteria, mycobacteria, parasites, fungi, yeast, viruses, or other microscopic organisms. An antimicrobial substance may be a microbistatic or microbicidal agent. As used herein, “microbistatic” means to slow or prevent the growth of microbes. For example, a microbistatic substance may interfere with protein production, DNA replication, cellular metabolism, etc. As used herein “microbicidal” means to kill microbes. The antimicrobial foamable compositions described herein may be used to kill or inhibit bacteria, mycobacteria, parasites, fungi, yeast, viruses, or other microscopic organisms. The foamable compositions of the invention are particularly adapted to be microbicidal with respect to MRSA.

The term “antimicrobial amount” refers to an amount of a substance that inhibits the growth of or kills bacteria, mycobacteria, parasites, fungi, yeast, or viruses. For example, an antimicrobial amount of thyme oil is an amount of thyme oil capable of inhibiting or killing bacteria, particularly MRSA, mycobacteria, parasites, fungi, yeast, or viruses.

As used herein, the term “antibacterial” refers to a substance that inhibits bacterial growth (“bacteriostatic”) or kills bacteria (“bacteriocidal”). The terms “antibiotic” and “antibiotic amount” as used herein refers to a substance or an amount, respectively, that inhibits bacterial growth, inhibits bacterial virulence, or kills bacteria. Antibiotics include natural, synthetic, or semi-synthetic compounds. The foamable compositions described herein may be used as an antibacterial agent effective against one or more bacterial strains including, but not limited to, Staphylococcus, such as MRSA, Streptococcus, Pseudomonas, Helicobacter, Salmonella, and Enterococcus.

As used herein, the term “antifungal” refers to a substance that inhibits fungal growth and/or replication (“fungistatic”) or kills fungi (“fungicial”). Examples of fungi include Trichophyton, Microsporum, and Epidermophyton. The term “antifungal” is an FDA regulated term defined as a drug that inhibits the growth and reproduction of fungal cells and decreases the number of fungi present. The term “fungus” as used herein is defined according to the FDA definition of any of a large division of plants, including dermatophytes, yeasts, and molds, characterized by a simple cell structure and the absence of chlorophyll. Fungal infections can cause skin disorders such as jock itch, ring worm, and athlete's foot.

As used herein, the term “antiviral” refers to a substance that inhibits viral growth and/or replication (“virustatic”) or kills viruses (“virucidal”).

The foamable composition may be used as at least one of a microbistatic, microbicidal, bacteriostatic, bacteriocidal, fungistatic, fungicidal, virustatic, and virucidal substance. Specifically, embodiments of the foamable composition have demonstrated high bactericidal efficacy. More specifically, the foamable composition are remarkably effective against drug-resistant bacteria, including several strains of MRSA. Efficacy (i.e., the kill rate) was determined by measuring the percent reduction of MRSA bacteria at a given time following application of the foamable composition to a surface (animate or inanimate) infected with MRSA. The microbial reduction assays are described in the Examples below. The bactericidal efficacy of the foamable compositions was determined from the MRSA kill rate.

As used herein, the term “drug resistance” refers to the ability of a microorganism to withstand the effects of a drug. “Antibiotic resistance” refers to a specific type of drug resistance in which the microorganism is able to withstand the effects of an antibiotic. Antibiotic resistance may evolve naturally or artificially. Bacteria that have developed resistance to a certain antibiotic are referred to as “drug resistant bacteria.” If a bacteria is resistant to several different compounds and/or a class of antibiotics, it is called “multi-resistant” or “multi-drug resistant.” For example, MRSA is a multi-drug resistant strain of the bacteria Staphylococcus aureus. The term “antimicrobial resistance” refers to the ability of a microorganism to withstand an antimicrobial substance. For example, drug resistant strains have been reported for numerous microbes in addition to Staphylococcus spp., including but not limited to, Enterococcus spp., Streptococcus spp., Escherichia spp., Mycobacterium spp., Salmonella spp., Campylobacter spp., Acinetobacter spp., Saccharomyces spp., Candida spp., Cryptococcus spp., and Pseudomonas spp. The abbreviation spp. is used to mean species in the designated genus.

Without being bound by theory, it is believed that natural essential oils that have not been refined or adulterated contain non-principal constituents that contribute to the environmental and human health and safety profiles of the compositions, including their antimicrobial properties. As the mechanism of antimicrobial activity is most often unknown for natural essential oils, additional refining beyond the extraction from a plant source of natural whole oil may modify the environmental and health and safety profiles of the essential oil in a negative fashion and possibly promote microbe resistance. Natural essential oils are used as the sole or principal antimicrobial agents in the foamable compositions described herein. In particular, thyme oil and/or origanum oil are used as natural antimicrobial agents in the foamable compositions. As used herein, thyme oil as the sole or principal antimicrobial component includes the essential oil of thyme and all of its naturally-occurring constituents, thymol and thymol derivatives.

The main chemical components of natural thyme oil extracted from Thymus vulgaris include α-thujone, α-pinene, camphene, β-pinene, p-cymene, α-terpinene, linalool, borneol, terpinen-4-ol, β-caryophyllene, thymol, and carvacrol. Thymol and carvacrol are monoterpene phenol compounds. Thymol is the principal component of natural thyme oil. Generally, natural thyme oil, on average, has approximately 35-40% thymol and thymol derivatives, and about 3% to about 7% carvacrol and carvacrol derivatives. Representative herbs from which thyme oil may be obtained include, but are not limited to, Thymus vulgaris, Thymus serpyllium, Thymus capitatus, Thymus mastichina and Thymus zygus.

Natural origanum oil generally has about 60% carvacrol and carvacrol derivatives, and about 3% to about 7% thymol and thymol derivatives. The phenolic compounds (e.g., thymol and thymol derivatives) present in origanum oil can also provide antimicrobial activity. Representative herbs from which origanum oil may be obtained include, but are not limited to, Origanum vulgar and Origanum dictamnus.

In one embodiment, the foamable composition contains an antimicrobial amount of thyme oil. The amount can be about 0.01% w/v to about 0.5% w/v, about 0.01% w/v to about 0.2% w/v, about 0.01% w/v to about 0.05% w/v, or, in an especially preferred embodiment about 0.016% w/v thyme oil.

In another embodiment, the foamable composition further comprises botanical thymol crystals. The amount can be about 0.01% w/v to about 0.5% w/v, about 0.01% w/v to about 0.1% w/v, or about 0.032% w/v.

In one embodiment, the foamable composition further comprises origanum oil. The amount can be about 0.01% w/v to about 0.2% w/v, about 0.01% w/v to about 0.05% w/v, or in an especially preferred embodiment about 0.016% w/v.

The foamable compositions described herein contain one or more surfactants, e.g., an alkyl polyglucoside surfactant. The alkyl polyglucoside surfactants, when mixed with the essential oils and water, help to form a stable macroemulsion or microemulsion of the foamable composition. The foamable compositions can contain an alkyl polyglucoside surfactant in an amount of about 0.01% v/v to about 10% v/v, about 0.5% v/v to about 8% v/v, or in an especially preferred embodiment about 2% v/v. Non-limiting examples of alkyl polyglucosides include capryl glucoside (Glucopon® 215CS UP), decyl glucoside (Glucopon®225DK), coco-glucoside (Glucopon® 425-N), lauryl glucoside (Glucopon® 625 UP), an aqueous solution of alkyl polyglucosides based on fatty acid alcohol C9-C11 (APG® 325N), and sodium laureth sulfate & lauryl glucoside & cocoamidopropyl betaine (Plantapon® 611L).

In one embodiment, the alkyl polyglucoside surfactant is a sulfonated surfactant, which renders the surfactant anionic. Nonlimiting examples of a sulfonated alkyl polyglucoside surfactant include sodium decylglucosides hydroxypropyl sulfonate (Suga®Nate 100), sodium decylglucosides hydroxypropyl sulfonate and sodium laurylglucosides hydroxypropyl sulfonate (Suga®Nate 124), and sodium laurylglucosides hydroxypropyl sulfonate Suga®Nate 160 (Suga®Nate 160).

One advantage to using alkyl polyglucoside surfactants, e.g., a sulfonated alkyl polyglucoside, over other types of surfactants is to maintain environmental and human health and safety profiles. Additionally, alkyl polyglucoside surfactants display excellent foaming characteristics and cause low to no irritation to the skin and eyes. Consequently, a foam comprising a non-irritating surfactant, such as an alkyl polyglucoside, may allow for improved treatment of wounds.

Additionally, the foamable composition described herein contains a source of divalent copper ions. Sources of divalent copper ions include, but are not limited to, cupric salts and copper salts such as copper sulfate, copper chloride, copper nitrate, copper acetate, cupric carbonate, cupric acetate, cupric citrate, cupric potassium chloride, cupric bromide, cupric chloride, cupric fluoroborate, cupric hydroxide, cupric nitrate, cupric oxide, and cupric sulfide. Copper peptide complexes are also a source of divalent copper ions.

In one embodiment, the source of divalent copper ions may be a copper peptide complex. The copper peptide complex can be present in an amount of from about 0.001% w/v to about 0.5% w/v, about 0.005% w/v to about 0.05% w/v, or in a preferred embodiment about 0.008% w/v. Copper peptides are small fragments of protein that have an affinity for copper. The peptide of the complex may comprise amino acids in the naturally occurring L-configuration, the D-configuration, or a mixture of D- and L-amino acids. The ratio of peptide molecule to copper may be either 1:1 or 2:1. Copper peptides may contribute to tissue regeneration, and in particular, the healing of wounds and skin lesions. Importantly, recent studies have suggested that copper peptides may reduce or eliminate various forms of skin irritation. See, Prickart, L., Skin Remodeling with Copper Peptides. Cosmetics and Medicine (Russia), 2004. Consequently, a foamable composition comprising a copper peptide complex may improve treatment of open wounds. Non-limiting examples of copper peptide complexes, such as copper (II) complexes, include glycyl-histidyl-lysine:copper (aka copper PCA), glycyl-(3-methyl)histidyl-lysine:copper, alanyl-histidyl-lysine:copper, alanyl-(3-methyl)histidyl-lysine:copper, glycyl-histidyl-phenylalanine:copper, glycyl-(3-methyl)histidyl-phenylalanine:copper, alanyl-histidyl-phenylalanine:copper, alanyl-(3-methyl)histidyl-phenylalanine:copper, glycyl-histidyl-lysyl-phenylalanyl-phenylalanyl:copper, glycyl-(3-methyl)histidyl-lysyl-phenylalanyl-phenylalanyl:copper, valyl-histidyl-lysine:copper, glycyl-arginyl-lysine:copper, glycyl-(5-methyl)arginyl-lysine:copper, alanyl-arginyl-lysine:copper, alanyl-(5-methyl)histidyl-lysine:copper, and glycyl-arginyl-phenylalanine:copper. In a particularly preferred embodiment, the foamable composition comprises copper PCA. Combinations of any of the foregoing and other copper peptide complexes may be used.

The foamable composition may be optionally provided as a concentrate wherein the amount of carrier present in the composition is reduced. For example, the amount of water present in the composition may be reduced. The concentrated composition may be reconstituted as desired by the addition of water at the time of use. The concentrated composition may be provided as about a 1:10, 1:50, or 1:100 concentrate, for example.

The foamable composition may optionally include one or more other ingredients to improve the aesthetic or other beneficial properties. Such optional ingredients may include fragrances, deodorizers, coloring agents, degreasing compounds, penetration enhancers, or any other inactive ingredient commonly used in topical cosmetics or pharmaceuticals. These optional ingredients, however, should be compatible with the core components of the antimicrobial foamable composition. The addition of these optional ingredients to the foamable composition should not negatively affect the efficacy or the environmental and human health and safety profiles of the foamable composition.

In another embodiment, the foamable composition may further comprise at least one fragrance. The fragrance can be present in an amount of about 0.01% v/v to about 5.0% v/v, about 0.01% v/v to about 1.0% v/v, or about 0.01% v/v to about 0.5% v/v. Non-limiting examples of a fragrance include vanilla, lavender, rose, rosemary, violet leaf, ginger, bergamot, spearmint, mint, eucalyptus, lime, blood orange, tangerine, grapefruit, lemon, lemongrass, petitgrain, litsea cubeba, pine, cedarwood, rosewood, chamomile, magnolia, and geranium. In preferred embodiments, the foamable composition includes one or more of: blood orange, vanilla, lavender, ginger, bergamot, spearmint, and lime.

It should be noted that the foamable compositions described herein differ from other soap products that include thyme oil merely as a fragrance. In contrast, the unique formulation of the thyme oil foamable compositions described herein achieve antimicrobial activity. Moreover, the foamable compositions achieve high antimicrobial activity with low levels of thyme oil. In particular, the thyme oil in combination with particular glucoside surfactants provide a very effective antimicrobial composition, particularly when used to kill MRSA.

In another embodiment, the foamable compositions may further comprise at least one antioxidant. The antioxidant can be present in an amount of about 0.001% v/v to about 0.5% v/v, about 0.001% v/v to about 0.1% v/v, or about 0.008% v/v to about 0.01% v/v. Antioxidants are molecules that neutralize free radicals in the body. Free radicals are a natural product of standard reactions within the cell and are important in many biological processes. However, free radicals can also damage cells and cause abnormal cellular function (e.g., collagen breakdown). Antioxidants may help prevent the damage that is caused by exposure to free radicals. In addition, antioxidants may also help stimulate the production of collagen fibers which is essential to maintaining and repairing the skin. Recent studies have suggested that antioxidants and the redox environment of the wound may play an important role in wound healing. See, Sen, C. K., The general case for redox control of wound repair. Wound Repair Regen., 11(6):431-8, 2003; Watt J, et al., Clin Sci (Lond)., 114(4):265-73, 2008. Consequently, a foamable composition comprising an antioxidant may improve treatment of open wounds and skin lesions. Non-limiting examples of an antioxidant include white tea extract, green tea extract, GHK copper peptides (e.g., copper PCA), vitamin C (L-ascorbic acid), grape seed extract, marine algae (e.g., Haematococcus algae), vitamin E, lycopene, bioflavanoids, blueberry extract, blackberry extract, pomegranate extract, beta carotene, idebenone, and coenzyme Q10. In preferred embodiments, the foamable comprises white tea extract and/or copper PCA.

The foamable composition may further comprise a natural moisturizer. The natural moisturizer can be present in an amount of about 0.01% v/v to about 5.0% v/v, about 0.05% v/v to about 1.0% v/v, or about 0.1% v/v. The natural moisturizer can be, e.g., a humectants that is mild and non-irritating. Non-limiting examples of suitable moisturizers include hydrolyzed wheat protein and hyaluronic acid (Cromoist® WHYA), hydrolyzed oats (Cromoist® O-25), hydrolyzed wheat protein and hydrolyzed wheat starch (Cropeptide® W), hydrolyzed silk (Ciosilk® 10,000), silk amino acids (Crosilk® Liquid), Cocodimonium hydroxypropyl silk amino acids (Crosilkquat®), collagen amino acids (Crotein® CAA/SF), hair keratin amino acids and sodium chloride (Crotein® HKP), keratin amino acids (Crotein® HKP/SF), collagen amino acids (Crotein® MCAA), hydrolyzed milk protein (Hydrolactin® 2500), hydrolyzed soy protein (Hydrosoy® 2000), hydrolyzed wheat protein (Hydrotriticum® 2000), wheat amino acids (Hydrotriticum® WAA), and hydrolyzed wheat protein (TritisolT®). In a preferred embodiment, the foamable composition comprises a hydrolyzed oat moisturizer. Combinations of any of the foregoing moisturizers may be employed.

In one embodiment, the foamable composition may further comprise aloe vera extract. The aloe vera extract can be present in an amount of about 0.1% v/v to about 5.0% v/v, about 0.1% v/v to about 1.0% v/v, or about 0.25% v/v aloe vera extract. Aloe vera is useful for treating skin conditions because it can reduce inflammation and pain to promote wound healing, and moisturize to treat dry skin conditions. In addition, aloe vera may also be a source of antioxidants useful in promoting and maintaining healthy skin as discussed above.

In another embodiment, the foamable composition may further comprise at least one co-surfactant. The co-surfactant can be present in amount of about 0.1% v/v to about 10% v/v, about 0.5% v/v to about 8% v/v, or about 5% v/v. The co-surfactant further helps to solubilize and disperse the essential oil(s) in water. The co-surfactants may be anionic, cationic, zwitteronic, or nonionic surfactants. Non-limiting examples of suitable co-surfactants include sodium laurel ether sulphate, sodium laurel sulphate, sodium lauryl sulfate, sarcosinates, yucca, naturally-derived sulfosuccinate, betaine, sultaine, propionate, acetate, amine oxide, naturally-derived ammonium chloride, geminis, carboxylate, and alcohol ethoxylate. Preferably, the co-surfactant is a biobased surfactant, which is a surfactant that is not principally derived from a petrochemical feedstock.

The foamable composition preferably employ water as a carrier. In one embodiment the pH of the foamable composition is in a range of about 0.2 to about 8.0, most preferably the pH is the range of about 3.5 to about 6. In one embodiment, the composition may further comprise at least one pH modifying agent, such as citric acid or sodium citrate. The pH modifying agent can be present in an amount of about 0.1% w/v to about 5% w/v, about 0.1% w/v to about 0.5% w/v, about 0.4% w/v, or about 0.2% w/v.

The foamable compositions described herein may be classified by the FDA as an over-the-counter antiseptic drug product. An “antiseptic” is defined by the FDA as a product that is used to prevent infection by killing or inhibiting the growth of microorganisms. Antiseptics include “consumer antiseptics,” which are defined by the FDA as a class of antimicrobial drug products marketed or proposed for use by the general public in a variety of settings. Consumer antiseptics include antibacterial soaps, hand sanitizers, and antibacterial wipes. “Healthcare antiseptics” are products used in a hospital or healthcare setting, where the risk of infection is greater. Healthcare antiseptics include handwashes, preoperative skin preparations, surgical hand scrubs, and hand sanitizers for use primarily in hospitals, clinics, doctor's offices, outpatient setting, nursing homes, and the like. The 1994 FDA Tentative Monographs propose that both consumer and healthcare personnel antiseptics achieve the efficacy criteria of 2 log₁₀ bacterial reduction after the first use. The antimicrobial foamable compositions described herein have demonstrated efficacy greater than the standards for both consumer and healthcare products as proposed in the most current FDA monograph (1994) (see Example 1, Table 3; Example 2, Table 5; Example 3, Table 7).

The foamable composition may also be used to treat a number of other skin infections, including, but not limited to, impetigo, folliculitis, boils, eethyma, erysipelas, cellulitis, jock itch, acne, and athlete's foot. Many current acne treatment use harsh chemicals (e.g., salicylic acid) that can irritate skin. The foams described herein are a gentle, natural, and effective product for treating or preventing acne.

The foamable compositions may also be useful as an antimicrobial substance to prevent or treat microbial infections of other tissues including the rectum, vagina, penile urethra, mucosal membranes, and oral cavity.

In one embodiment, the foamable composition may be used as a “therapeutic bandage” wherein the composition is applied to the skin and allowed to spread and/or cover the affected area. Microbes often enter the body through a wound, resulting in a more serious infection. As used herein, a “wound” refers to an injury to the skin, tissue, or external surface of the body often as a result of physical trauma that causes disruption of the normal continuity of structure. As used herein, an “open wound” refers to a type of wound in which in the skin is torn, cut, pierced, punctured, or otherwise impaired so as to expose the injury and the underlying tissue. An open wound is at high risk for infection. In particular, Staph and MRSA infections are often associated with entry into the body through a wound (e.g., as a result of injury or surgery). The foamable composition may be less irritating to wounds, thus improving patience compliance and maximizing the therapeutic effect. Alternatively, upon further application of a mechanical force (e.g., rubbing the composition onto the surface), the foam freely spreads on the surface and is rapidly absorbed. In addition, the foam may be useful to prevent and/or treat secondary infections accompanying skin structure damage, such as in cuts, wounds, burns, and lesions. In all such cases, the antimicrobial foamable composition is easy to use and spreads easily, covering the affected area without causing pain.

The antimicrobial foamable composition may removed (e.g., wiped or rinsed) from the surface after application. Alternatively, the foam may be left on the surface following application. In one embodiment, the foamable composition may be applied to mammalian skin, more preferably to human skin. Sustained contact with the area bearing the microorganisms may ensure a higher killing rate and continuous germ control for extended periods of time. Further, since the foamable compositions do not require wiping or rinsing to remove any antimicrobial residues, the present compositions are convenient and easy to use. In some instances, it may be desirable to cover the application area with a sterile bandage or gauze. For example, the composition may be applied to the skin and/or wound infected with MRSA and covered with a sterile bandage. Alternatively, the foam may first be applied to a bandage, gauze, or other applicator that is subsequently contacted to the skin and/or wound. The bandage in combination with the foamable composition may function to further protect the infected area.

The foamable compositions are preferably alcohol-free and preferably do not contain synthetic chemicals commonly found in cosmetics and pharmaceuticals (e.g., petrochemicals and parabens). Further, the compositions are non-corrosive, non-flammable, non-reactive, readily biodegradable, and have a very low volatile organic compound level of less than 1%.

The foamable composition may be topically applied for a duration of seconds to hours. For example, the foam may remain in contact with the surface (i.e., exposure time) about 15 seconds to about 300 seconds, about 5 minutes to about 60 minutes, about one hour to about 24 hours, or longer. Adequate exposure time may result in at least 99.9% of microbes killed in 15 seconds. More preferably, after an exposure time of 15 seconds, 99.99% of MRSA was killed. (See Example 2, Table 5 and Example 3, Table 7). The foamable composition may be applied one or more times a day (e.g., once a day, twice a day, three times a day) or as needed. The foam can be applied directly to the surface, such as the skin and/or wound.

One or more additional products may be co-administered, either within the foamable composition or separately administered. For example, the foam may be co-administered with another antimicrobial or antibacterial product, a topical analgesic, or an antipyretic. Non-limiting examples of an antimicrobial product include clindamycin, salicylic acid, gramicidin, neomycin, and polymyxin. Non-limiting examples of a topical analgesic include capsaicin, lidocaine, and methyl salicylate.

The particular combination of components used in the foamable compositions provides a stable composition. Preferably, the foams will have a shelf life of at least 2 years.

The foamable composition may be formulated by conventional procedures known in the art. For example, the foamable composition can be formulated by combining thyme oil, an alkyl polyglucoside surfactant, and water together. The combined ingredients are then agitated or mixed until a macroemulsified or microemulsified solution of thyme oil is formed.

Individuals in need of the foamable composition can be identified by one or more risk factors for developing a MRSA infection. The risk factors for healthcare-associated versus community-associated MRSA may differ due to the different settings. Risk factors for HA-MRSA include visiting a hospital or long-term care facility as a visitor, patient, resident, or worker. Individuals who are on dialysis, are catheterized, or have feeding tubes or other invasive devices (e.g., catheters and intravenous lines) are more likely to develop a MRSA infection. Older adults (about 65 years of age or older) or individuals with weakened immune systems, burns, surgical wounds, or serious underlying health problems that are hospitalized are also at increased risk for infection with HA-MRSA. A 2007 report from the Association for Professionals in Infection Control and Epidemiology estimates 1.2 million hospital patients are infected with MRSA each year in the United States. MRSA is even more prevalent in long-term care facilities than in hospitals. It should also be noted that recent treatment with a course of antibiotics such as fluoroquinolones (e.g., ciprofloxacin, ofloxacin, or levofloxican) or cephalosporins may increase the risk of MRSA infection in some patients.

The main risk factors for CA-MRSA include age, participating in contact sports, sharing towels or athletic equipment, a weakened immune system, living in crowded or unsanitary conditions, and association with healthcare workers. CA-MRSA can be particularly dangerous in children (up to about 16 years of age), because they are more likely to develop dangerous forms of pneumonia than adults. In particular, those individuals with weakened immune systems, such as children and individuals infected with HIV/AIDS, may be more likely to develop severe MRSA infections. CA-MRSA outbreaks have been reported in public gyms as well as among both amateur and professional sports teams. In addition, outbreaks of CA-MRSA have occurred in military training camps. An individual who has one or more of these risk factors for CA-MRSA or HA-MRSA is at increased risk for developing MRSA infection, and may benefit from the antimicrobial foamable compositions of the present invention.

The foamable composition may be formulated to be dispersed from a dispenser, such as a pump or a bottle. The dispenser may be manual or automatic, such that the dispenser may be a hands-free dispenser that provides an amount of foam without having to press a button, lever, etc. thereby further minimizing the spread of bacteria and other microbes. Exemplary locations for a foamable composition dispenser include, without limitation, homes, schools, gyms, hospitals, long-term care facilities, day care facilities, dormitories, and military camps.

The foamable composition should be of a consistency as to allow it to flow through the dispenser, such that air bubbles can be trapped, and an acceptable foam produced. The quality of the foam may include a rich and creamy appearance, very small bubble size, and the ability to retain creaminess upon spreading on the skin such that it does not become watery.

It is understood that the invention is not limited to the particular methodology, protocols, and reagents, etc., described herein, as these may vary as one of ordinary skill in the art will recognize. The terminology used herein is used to describe particular embodiments only, and is not intended to limit the scope of the invention. As used herein and in the appended claims, the singular forms “a,” “an,” and “the” include the plural reference unless the context clearly dictates otherwise. For example, a reference to “a fragrance” is a reference to one or more fragrances.

Numerical ranges recited herein include all circumscribed ranges i.e., those where the upper and lower values of such ranges are numbers that fall within the broad range.

### EXAMPLES

Antimicrobial foamable compositions were prepared, having ingredients in the amount by volume (% v/v) specified below.

Formulations of the foamable composition. Four formulations of the foamable composition were prepared having the ingredients specified in Table 1 below.

TABLE 1
Formulations.

| | Formulations | | | |
|---|---|---|---|---|
| Ingredients | 1 | 2 | 3 | 4 |
| Thymol Crystals - botanical | 0.032 | 0.032 | 0.032 | 0.032 |
| (% w/v) | | | | |
| Thyme Oil 50% (% w/v) | 0.016 | 0.016 | 0.016 | 0.016 |
| Origanum Oil (% w/v) | 0.016 | 0.016 | 0.016 | 0.016 |
| Fragrance (total) (% v/v) | 0.6 | 0.3 | 0.226 | 0.32 |
| blood orange | 0.1 | | | |
| vanilla | 0.5 | | | |
| lavender | | 0.2 | | |
| ginger | | 0.1 | 0.167 | |
| bergamot | | | 0.059 | |
| spearmint | | | | 0.15 |
| lime | | | | 0.17 |
| Suga ® Nate 100 (% v/v) | 3.0 | 3.0 | 2.0 | 3.0 |
| SLS (Stepan) (% v/v) | 5.0 | 5.0 | 5.0 | 5.0 |
| White Tea (% v/v) | 0.01 | 0.01 | | 0.001 |
| Citric Acid USP (% w/v) | 0.21 | 0.21 | 0.21 | 0.21 |
| Sodium Citrate USP (% w/v) | 0.4 | 0.4 | 0.4 | 0.4 |
| Cromoist ® O-25 (% v/v) | 0.1 | 0.1 | 0.1 | 0.1 |
| Aloe Vera Extract 10X (% v/v) | 0.25 | 0.25 | 0.25 | 0.25 |
| Copper PCA (% w/v) | 0.008 | 0.008 | 0.008 | 0.008 |
| Deionized water (% v/v) | 90.358 | 90.658 | 91.741 | 90.647 |
| TOTAL | 100.0 | 100.0 | 100.0 | 100.0 |

Time Kill Test Assay for Antimicrobial Agents.

A suspension of bacterial cells was exposed to the test substance for specified contact times. After exposure, an aliquot of the suspension was transferred to a neutralizer and assayed for survivors. Appropriate purity, sterility, microorganism population and neutralization controls were performed.

The test organisms were Community Acquired Methicillin Resistant Staphylococcus aureus—CA-MRSA (NRS384) (Genotype USA300) or Staphylococcus aureus—MRSA (ATCC 33592). The test organisms were purchased from the NARSA Contracts Administrator or the American Tissue Type Culture Collection (ATCC), respectively, by ATS Labs.

Inoculum Preparation: Using a stock culture of the test organism, a culture of the test organism was streaked onto the culture medium of tryptic soy agar containing 5% sterile sheep blood. The bacterial cultures were incubated for 24-48 hours at 35-37° C. (alternate or extended incubation may be required for certain strains). A sufficient amount of organism growth was transferred into a sterile diluent to yield a uniform suspension of approximately 1×10⁸ CFU/mL. (CFU is colony forming unit.) Most bacterial strains approximately matched a 0.5 McFarland standard. Cultures were further adjusted as needed. Antimicrobial susceptibility testing was performed utilizing a representative culture from the day of testing to verify the antimicrobial resistance pattern stated.

An organic soil load was added to the test culture on request. For example, a 0.1 mL aliquot of FBS was added to 1.9 mL of broth culture to yield a 5% fetal bovine serum soil load.

Preparation of Test Substance. The test substance was prepared according to the directions supplied. A 9.9 mL aliquot of the prepared test substance was transferred to a sterile vessel (e.g., glass tube, stomacher bag, etc.) for testing procedures. The test substance was used within 3 hours of preparation if additional preparation was required by ATS Labs.

Test Substance Exposure: A 0.1 mL aliquot of the standardized inoculum was added to the test substance representing the start of the test exposure. The inoculated test substance was immediately mixed thoroughly using a laboratory stomacher, vortex mixer or other applicable method. The inoculated and mixed test substance was held at the specified temperature. If the requested exposure temperature lies outside of achievable ambient conditions, the test substance was equilibrated in a water bath (or other appropriate device) to equilibrate to the desired exposure temperature. An inoculum of 0.1 mL of the organism suspension was added to 9.9 mL of the test substance and vortex mixed. The test mixture was exposed for 15, 30, 60, 120 and 300 seconds at ambient temperature (21° C.).

Subculture: At each specified exposure time, a 1.0 mL aliquot of the inoculated est substance was transferred to 9 mL of neutralizer broth. Four additional 1:10 dilutions in Butterfield's Buffer were prepared. Using a standard microbiological spread plate count procedure, 1.0 mL aliquots of each dilution (10⁻¹⁰-10⁻⁴) were plated in duplicate to the appropriate recovery media. A 5.0 mL aliquot of the neutralized sample was transferred to a sterile 0.45 μm filter apparatus system pre-wetted with 10 mL sterile diluent (e.g., 0.85% sterile saline). The sample was filter concentrated and the filter rinsed using ≧50 mL sterile diluent (e.g., 0.85% sterile saline). The filter was aseptically removed and placed on the surface of the recovery agar medium.

Incubation and Observation: All bacterial subculture plates were incubated for 48±4 hours at 35-37° C. Subculture plates were optionally refrigerated at 2-8° C. for ≦3 days prior to examination. Following incubation, the test and controls were visually examined for growth. Agar plates were enumerated and recorded. Log and percent reductions were determined for each time point. Representative subcultures demonstrating growth were appropriately examined for confirmation of the test organism.

Test Population Control: In a similar manner as the culture inoculum was added to the test substance, an equivalent volume (0.1 mL) of each inoculum was added to 9.9 mL of Butterfield's (same volume as the test substance). This suspension was neutralized as in the test procedure. This suspension was serially diluted and appropriate dilutions were plated using standard microbiological techniques. Following incubation, the organism plates were observed to enumerate the concentration of the test organism present in the test substance at the time of testing (time analysis). The acceptance criterion for this study control is growth and the value is used for calculation purposes only.

Purity Control. A streak plate for isolation was performed on the organism culture. Following incubation, they were examined to confirm the presence of a pure culture. The acceptance criterion for this study control is a pure culture demonstrating colony morphology typical of the test organism.

Initial Suspension Population Control. The prepared test organism suspension was serially diluted and plated using standard microbiological techniques. Following incubation, the organism plates were observed to enumerate the concentration of the test organism inoculated into the test substance at the time of testing. The acceptance criterion for this study control is growth of ≧1.0×10⁶ CFU/mL.

Neutralizer Sterility Control. A representative sample of the neutralizer was incubated and observed. The acceptance criterion for this study control is lack of growth.

Organic Soil Sterility Control. The serum used for soil load was cultured, incubated, and observed for lack of growth. For example, 1.0 mL of the serum used for soil load was added to a tube of Fluid Thioglycollate, incubated, and observed for lack of growth. The acceptance criterion for this study control is lack of growth.

Neutralization Control. To simulate testing conditions. 9.9 mL of the test substance was inoculated with 0.1 mL Butterfield's Buffer in place of the test organism suspension (NC Suspension).

(1) Filtration Neutralization: A 1.00 mL aliquot of the NC Suspension was transferred to 9 mL neutralizing broth and mixed thoroughly. The control suspension (5.0 mL) was filter concentrated and the filter was rinsed as in the test procedure. An aliquot (1.0 mL) of an organism suspension containing approximately 100 CFU/mL was added to the filter apparatus and processed through the apparatus. An aliquot (1.0 mL) of the organism suspension was added to a second filter apparatus to be used as an inoculum population control and processed. The filters were aseptically transferred to recovery agar plates and incubated. The acceptance criteria for this study control requires the filtration neutralization control and corresponding population control results to be within 1.0 Log.

(2) Chemical Neutralization: A 1.0 mL aliquot of the NC Suspension was transferred to 9 mL neutralizing broth and mixed thoroughly. A 1.0 mL aliquot of the neutralized sample was then removed and discarded. To the neutralized sample, 1.0 mL of the organism suspension (containing approximately 1000 CFU/mL) was added and mixed thoroughly. An aliquot (1.0 mL) of the neutralized mixture was plated in duplicate and incubated. An inoculum population control was performed by adding 1.0 mL of the same organism suspension to 9 mL of Butterfield's Buffer, plating in duplicate, and incubating. The acceptance criterion for this study control requires the chemical neutralization control and corresponding population control by within 1.0 Log.

Data was analyzed using the calculations listed below.

$$Test Data CFU / mL = \frac{\begin{matrix}( avg . \# colonies found / plate @ dilution used ) \\ ( dilution factor ) ( volume neutralized solution )\end{matrix}}{( volume plated )}$$

$$Percent Reduction = [ 1 - ( test survivors / test population control ) ] \times 100$$

$$Log Reduction = {Log}_{10} ( test population control ) - {Log}_{10} ( test survivors )$$

The above described methodology is an example which may be adapted for testing the efficacies of the antimicrobial product as required by governing agencies. The examples below were carried out using standard, peer-reviewed methods developed by the American Society for Testing and Materials (ASTM). The ASTM methodology has been proposed as the FDA standard for efficacy in the Final Monograph for over-the-counter antibacterial drugs.

### Example 1

### Time Kill Test Assay Against CA-MRSA

Under the conditions of this study, the foamable composition of Formulation 1 demonstrated a 99.9% (3.735 Log₁₀) reduction of Community Acquired Methicillin Resistant Staphylococcus aureus—CA-MRSA (NARSA NRS384) (Genotype USA300) survivors after a 15 second exposure, a 99.999% (5.2 Log₁₀) reduction after a 30 second exposure, a >99.999% (>5.8 Log₁₀) reduction after a 60 second exposure, a >99.999% (>5.8 Log₁₀) reduction after a 120 second exposure and a >99.999% (>5.8 Log₁₀) reduction after a 300 second exposure when tested at ambient temperature (21° C.).

TABLE 2
Test Results.

| | Test Organism: Community | | | | |
|---|---|---|---|---|---|
| | Acquired Methicillin Resistant | | | | |
| | Staphylococcus aureus - CA-MRSA | | | | |
| | TIME EXPOSURE | | | | |
| | 15 | 30 | 60 | 120 | 300 |
| | seconds | seconds | seconds | seconds | seconds |
| DILUTION | Number of Survivors Recovered | | | | |
| 10⁻¹ | 10, 9 | 0, 0 | 0, 1 | 0, 0 | 0, 0 |
| 10⁻² | 0, 1 | 0, 0 | 0, 0 | 0, 0 | 0, 0 |
| 10⁻³ | 0, 0 | 0, 0 | 0, 0 | 0, 0 | 0, 0 |
| 10⁻⁴ | 0, 0 | 0, 0 | 0, 0 | 0, 0 | 0, 0 |
| Filtration of 5.0 mL at | 114 | 4 | 0 | 0 | 0 |
| 10⁰ dilution | | | | | |

A value of <1 was used in place for calculation purposes only.

TABLE 3
Calculated Data.

| | | Test | | | | |
|---|---|---|---|---|---|---|
| | | Population | | | | |
| | | Control | Number of | Log₁₀ | | |
| | Exposure | (CFU/mL)* | Survivors | Number of | Percent | Log₁₀ |
| Test Organism | Time | Log₁₀ | (CFU/mL)* | Survivors | Reduction | Reduction |
| Staphylococcus | 15 seconds | 1.24 × 10⁶ | 2.28 × 10² | 2.358 | 99.9% | 3.735 |
| aureus - CA-MRSA | 30 seconds | (6.093 Log₁₀) | 8 | 0.9 | 99.999% | 5.2 |
| | 60 seconds | | <2 | <0.3 | >99.999% | >5.8 |
| | 120 seconds | | <2 | <0.3 | >99.999% | >5.8 |
| | 300 seconds | | <2 | <0.3 | >99.999% | >5.8 |

*colony forming units per mL of test mixture

### Example 2

### Time Kill Test Assay Against MRSA

Under the conditions of this study, the foamable composition of Formulation 1 demonstrated a 99.99% (4.32 Log₁₀) reduction of Staphylococcus aureus—MRSA survivors after a 15 second exposure, a 99.999% (5.5 Log₁₀) reduction after a 30 second exposure, a >99.999% (>5.8 Log₁₀) reduction after a 60 second exposure, and a >99.999% (>5.8 Log₁₀) reduction after a 300 second exposure in the presence of a 5% fetal bovine serum organic soil load when tested at ambient temperature (20° C.).

TABLE 4
Test Results.

| | Test Organism: Staphylococcus aureus - MRSA | | | |
|---|---|---|---|---|
| | TIME EXPOSURE | | | |
| | 15 seconds | 30 seconds | 60 seconds | 300 seconds |
| DILUTION | Number of Survivors Recovered | | | |
| 10⁻¹ | 1, 0 | 0, 0 | 0, 0 | 0, 0 |
| 10⁻² | 0, 0 | 0, 0 | 0, 0 | 0, 0 |
| 10⁻³ | 0, 0 | 0, 0 | 0, 0 | 0, 0 |
| 10⁻⁴ | 0, 0 | 0, 0 | 0, 0 | 0, 0 |
| Filtration of | 30 | 2 | 0 | 0 |
| 5.0 mL at | | | | |
| 10⁰ dilution | | | | |

A value of <1 was used in place of zero for calculation purposes only.

TABLE 5
Calculated Data.

| | | Test | | | | |
|---|---|---|---|---|---|---|
| | | Population | | | | |
| | | Control | Number of | Log₁₀ | | |
| | Exposure | (CFU/mL)* | Survivors | Number of | Percent | Log₁₀ |
| Test Organism | Time | Log₁₀ | (CFU/mL)* | Survivors | Reduction | Reduction |
| Staphylococcus | 15 seconds | 1.26 × 10⁶ | 6.0 × 10¹ | 1.78 | 99.99% | 4.32 |
| aureus - MRSA | 30 seconds | (6.1 Log₁₀) | 4 | 0.6 | 99.999% | 5.5 |
| | 60 seconds | | <2 | <0.3 | >99.999% | >5.8 |
| | 300 seconds | | <2 | <0.3 | >9.999% | >5.8 |

*colony forming units per mL of test mixture

### Example 3

### Time Kill Test Assay Against MRSA

Under the conditions of this study, the foamable composition of Formulation 3 demonstrated a 99.99% (4.84 Log₁₀) reduction of Staphylococcus aureus—MRSA survivors after a 15 second exposure, a >99.999% (>5.8 Log₁₀) reduction after a 30 second exposure, a >99.999% (>5.8 Log₁₀) reduction after a 60 second exposure, and a >99.999% (>5.8 Log₁₀) reduction after a 300 second exposure in the presence of a 5% fetal bovine serum organic soil load when tested at ambient temperature (20° C.).

TABLE 6
Test Results.

| | Test organism: Staphylococcus aureus - MRSA | | | |
|---|---|---|---|---|
| | TIME EXPOSURE | | | |
| | 15 seconds | 30 seconds | 60 seconds | 300 seconds |
| DILUTION | Number of Survivors | | | |
| 10⁻¹ | 0, 1 | 0, 0 | 0, 0 | 0, 0 |
| 10⁻² | 0, 0 | 0, 0 | 0, 0 | 0, 0 |
| 10⁻³ | 0, 0 | 0, 0 | 0, 0 | 0, 0 |
| 10⁻⁴ | 0, 0 | 0, 0 | 0, 0 | 0, 0 |
| Filtration of | 10 | 0 | 0 | 0 |
| 5.0 mL at | | | | |
| 10⁰ dilution | | | | |

A value of <1 was used in place of zero for calculation purposes only.

TABLE 7
Calculated Data.

| | | | | | | |
|---|---|---|---|---|---|---|
| | | Test | | | | |
| | | Population | | Log₁₀ | | |
| | | Control | Number of | Number | | |
| | Exposure | (CFU/mL)* | Survivors | of | Percent | Log₁₀ |
| Test Organism | Time | Log₁₀ | (CFU/mL)* | Survivors | Reduction | Reduction |

| | | | | | | |
|---|---|---|---|---|---|---|
| Staphylococcus | 15 seconds | 1.38 × 10⁶ | 2.0 × 10¹ | 1.3 | 99.99% | 4.84 |
| aureus - MRSA | 30 seconds | (6.14 Log₁₀) | <2 | <0.3 | >99.999% | >5.8 |
| | 60 seconds | | <2 | <0.3 | >99.999% | >5.8 |
| | 300 seconds | | <2 | <0.3 | >99.999% | >5.8 |

*colony forming units per mL of test mixture

### Example 4

### Time Kill Test Assay Against MRSA

Under the conditions of this study, the foamable composition of Formulation 2 demonstrated a >99.999% (>5.9 Log 10) reduction of Staphylococcus aureus—MRSA survivors after a 30, 60, 120 and 300 second exposure when tested at ambient temperature (20° C.).

TABLE 8
Test results.

| | Test organism: Staphylococcus aureus MRSA | | | |
|---|---|---|---|---|
| | TIME EXPOSURE | | | |
| | 30 seconds | 60 seconds | 120 seconds | 300 seconds |
| DILUTION | Number of Survivors | | | |
| 10⁻¹ | 0, 0 | 0, 0 | 0, 0 | 0, 0 |
| 10⁻² | 0, 0 | 0, 0 | 0, 0 | 0, 0 |
| 10⁻³ | 0, 0 | 0, 0 | 0, 0 | 0, 0 |
| 10⁻⁴ | 0, 0 | 0, 0 | 0, 0 | 0, 0 |
| Filtration of | 0 | 0 | 0 | 0 |
| 5.0 mL at | | | | |
| 10⁰ dilution | | | | |

TABLE 9
Calculated Data.

| | | Test | | | | |
|---|---|---|---|---|---|---|
| | | Population | | Log₁₀ | | |
| | | Control | Number of | Number | | |
| | Exposure | (CFU/mL)* | Survivors | of | Percent | Log₁₀ |
| Test Organism | Time | Log₁₀ | (CFU/mL)* | Survivors | Reduction | Reduction |
| Staphylococcus | 30 seconds | 1.45 × 10⁶ | <2 | <0.3 | >99.999% | >5.9 |
| aureus - MRSA | 60 seconds | (6.161 Log₁₀) | <2 | <0.3 | >99.999% | >5.9 |
| | 120 seconds | | <2 | <0.3 | >99.999% | >5.9 |
| | 300 seconds | | <2 | <0.3 | >99.999% | >5.9 |

The examples given above are merely illustrative and are not meant to be an exhaustive list of all possible embodiments, applications or modifications of the invention. Thus, various modifications and variations of the described methods and systems of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes of carrying out the invention which are obvious to those skilled in the chemical arts or in the relevant fields are intended to be within the scope of the appended claims.

The disclosures of all references and publications cited above are expressly incorporated by reference in their entireties to the same extent as if each were incorporated by reference individually.

What is claimed is:

1. A foamable composition for killing MRSA on a surface contaminated therewith, comprising:
an antimicrobial amount of thyme oil of about 0.01% w/v to about 0.5% w/v;
a surfactant; and
a source of divalent copper ions.

2. The composition of claim 1, wherein the composition kills greater than 99.99% of methicillin-resistant Staphylococcus aureus in 15 seconds when applied to said surface.

3. The composition of claim 1, wherein the composition kills greater than 99.9% of methicillin-resistant Staphylococcus aureus in 15 seconds when applied to said surface.

4. The composition of claim 1, wherein the antimicrobial amount of thyme oil is about 0.01% w/v to about 0.2% w/v.

5. The composition of claim 1, wherein the composition further comprises origanum oil.

6. The composition of claim 5, wherein the origanum oil is present in an amount of about 0.01% w/v to about 0.2% w/v.

7. The composition of claim 1, wherein the composition further comprises thymol crystals.

8. The composition of claim 7, wherein the thymol crystals are present in an amount of about 0.01% w/v to about 0.5% w/v.

9. The composition of claim 1, wherein the composition further comprises at least one of a fragrance, an antioxidant, a humectant, and a co-surfactant.

10. The composition of claim 9, wherein the fragrance is selected from the group consisting of blood orange, vanilla, lavender, ginger, bergamot, spearmint, and lime.

11. The composition of claim 9, wherein the antioxidant is white tea extract.

12. The composition of claim 1, wherein the source of divalent copper ion is a copper peptide complex.

13. The composition of claim 12, wherein the copper peptide complex is copper PCA.

14. The composition of claim 1, wherein the surfactant is an anionic or a nonionic surfactant.

15. The composition of claim 1, wherein the surfactant is selected from the group consisting of alkyl polyglucoside, sodium lauryl sulfate, and sodium laureth sulfate.

16. A foamable composition for killing MRSA on a surface contaminated therewith, comprising:
0.016% w/v thyme oil
0.016% w/v origanum oil
0.032% w/v botanical thymol crystals
3.0% v/v alkyl polyglucoside surfactant
0.008% w/v copper PCA
water to 100%.

17. The composition of claim 16, wherein the composition is capable of killing greater than 99.99% of methicillin-resistant Staphylococcus aureus in 15 seconds when applied topically.

18. The composition of claim 16, wherein the composition is capable of killing greater than 99.9% of methicillin-resistant Staphylococcus aureus in 15 seconds when applied topically.

19. The composition of claim 16, wherein the composition further comprises at least one of white tea, citric acid, sodium citrate, co-surfactant, natural moisturizer, and aloe vera.

20. A method for killing MRSA on a surface contaminated therewith, comprising:
applying to said surface a foamable composition comprising an antimicrobial amount of thyme oil of about 0.01% w/v to about 0.5% w/v, a surfactant and a source of divalent copper ions; and
exposing the surface to the composition for an exposure time sufficient to kill methicillin-resistant Staphylococcus aureus.

21. The method of claim 20, wherein said surface comprises mammalian skin.

22. The method of claim 20, wherein said mammalian skin comprises human skin.

23. The method of claim 21, wherein the foamable composition is applied to an open wound.

24. The method of claim 21, wherein the exposure time is at least 15 seconds.

25. The method of claim 21, wherein the exposure time is at least 5 minutes.

26. The method of claim 21, wherein the exposure time is at least an hour.

27. The method of claim 21, wherein the foamable composition is applied at least once a day.

28. The method of claim 21, wherein the composition kills greater than 99.99% of methicillin-resistant Staphylococcus aureus in 15 seconds.

29. The method of claim 21, wherein the composition kills greater than 99.9% of methicillin-resistant Staphylococcus aureus in 15 seconds.

30. A kit, comprising:
an antimicrobial foamable composition for killing MRSA on a surface contaminated therewith, comprising an antimicrobial amount of thyme oil of about 0.01% w/v to about 0.5% w/v, a surfactant, and a source of divalent copper ions; and
a dispenser.

31. The kit of claim 30, wherein the composition kills greater than 99.99% of methicillin-resistant Staphylococcus aureus in 15 seconds.

32. The kit of claim 30, wherein the composition kills greater than 99.9% of methicillin-resistant Staphylococcus aureus in 15 seconds.
